(19) United States
(12) Patent Application Publication
(10) Pub. No.: US 2026/0275429 A1
(43) Pub. Date: Sep. 17, 2026

(54) METHOD FOR ASSESSING IMPACT OF TRANSGENIC PLANTS ON AGRICULTURAL BIODIVERSITY USING ENVIRONMENTAL DNA METABARCODING SEQUENCING

(71) Applicant: Nanjing Institute of Environmental Sciences, Ministry of Ecology and Environment, Nanjing (CN)

(72) Inventors: Laipan LIU, Nanjing City (CN); Biao LIU, Nanjing City (CN); Zhixiang FANG, Nanjing City (CN); Wenjing SHEN, Nanjing City (CN); Zhentao REN, Nanjing City (CN); Li ZHANG, Nanjing City (CN); Qi YU, Nanjing City (CN)

(21) Appl. No.: 19/329,819

(22) Filed: Sep. 16, 2025

(30) Foreign Application Priority Data
Mar. 13, 2025 (CN) 202510296095.4

(51) Int. Cl. C12Q 1/6895; G01N 1/34

(52) U.S. Cl. CPC C12Q 1/6895; G01N 1/34; C12Q 2600/13

(57) ABSTRACT

The present disclosure relates to a method for assessing an impact of a transgenic plant on agricultural biodiversity using environmental DNA (eDNA) metabarcoding sequencing. The method includes the following steps: collecting rhizosphere soil samples and phyllosphere surface attachment samples of a transgenic crop during the same period, and extracting deoxyribonucleic acid (DNA) from the samples; using the extracted DNA as a template, performing polymerase chain reaction (PCR) amplification with amplification primers; and purifying PCR amplification products, subjecting the purified PCR amplification products to high-throughput sequencing (HTS) to obtain sequences, comparing the obtained operational taxonomic unit (OTU) sequences with the nucleotide (nt) public database on the website of the National Center for Biotechnology Information (NCBI) to identify arthropod species in the samples and calculate biodiversity-related indices, thereby assessing the impact of the transgenic crop on agricultural biodiversity.

### CROSS-REFERENCE TO RELATED APPLICATIONS

This application claims priority to Chinese Patent Application No. 202510296095.4, filed on Mar. 13, 2025, which is incorporated herein by reference in its entirety.

### REFERENCE TO AN ELECTRONIC SEQUENCE LISTING

The instant application contains a Sequence Listing which has been submitted electronically in XML format and is hereby incorporated by reference in its entirety. Said Sequence Listing XML, created on Sep. 10, 2025, is named METHOD FOR ASSESSING IMPACT OF TRANSGENIC PLANTS ON AGRICULTURAL BIODIVERSITY USING ENVIRONMENTAL DNA METABARCODING SEQUENCING.xml and is 2,999 bytes in size.

### TECHNICAL FIELD

The present disclosure belongs to the technical field of molecular ecology. Specifically, the present disclosure relates to a method for assessing an impact of a transgenic plant on agricultural biodiversity using environmental DNA (eDNA) metabarcoding sequencing.

### BACKGROUND

Large-scale cultivation of transgenic crops represents a significant strategy in modern agriculture to address global food security challenges. Insect-resistant transgenic crops, such as Bacillus thuringiensis (Bt) insect-resistant transgenic crops expressing Bt insecticidal proteins, indirectly protect biodiversity by reducing pesticide usage. Furthermore, high-yield transgenic crops may mitigate the encroachment of agricultural expansion on natural ecosystems by increasing yield per unit area; however, their ecological safety remains a subject of considerable concern. Part of this controversy stems from the limitations of traditional ecological methods in biodiversity assessment. Consequently, there is a pressing need for more precise and efficient evaluation systems.

Traditional biodiversity survey methods exhibit significant limitations in assessing the ecological impact of transgenic crops. Species inventories rely on field collection and taxonomic identification by specialized personnel, which restricts spatiotemporal coverage, incurs high costs, and struggles to capture the temporal dynamics of cryptic soil fauna and small, highly mobile species. Accurate determination of animal community composition and abundance cannot be achieved. Although existing molecular techniques have improved efficiency, they commonly suffer from genetic marker selection bias. Ribosomal ribonucleic acid (RNA) genes (e.g., 16S/18S ribosomal RNA (rRNA)) demonstrate a species-level identification success rate of less than 40% within the animal kingdom. Furthermore, while most studies focus on a single ecological niche (e.g., soil or air samples), a comprehensive assessment of the impact of transgenic crops on biodiversity requires a broader approach. These shortcomings render traditional methods inadequate for meeting the demands of simultaneous multi-interface and multi-taxa monitoring, and they also fail to differentiate the response differences between closely related species and cryptic species to transgenic crops.

### SUMMARY

The objective of the present disclosure is to provide a method for assessing an impact of a transgenic plant on agricultural biodiversity using eDNA metabarcoding sequencing, addressing the aforementioned problems.

The present disclosure achieves the above objective through the following technical solutions:

A method for assessing an impact of a transgenic plant on agricultural biodiversity using eDNA metabarcoding sequencing, including the following steps:
collecting rhizosphere soil samples and phyllosphere surface attachment samples of a transgenic crop during the same period, and extracting deoxyribonucleic acid (DNA) from the samples;
using the extracted DNA as a template, performing polymerase chain reaction (PCR) amplification with amplification primers; and
purifying PCR amplification products, subjecting the purified PCR amplification products to high-throughput sequencing (HTS) to obtain sequences, comparing the obtained operational taxonomic unit (OTU) sequences with the nucleotide (nt) public database on the website of the National Center for Biotechnology Information (NCBI) to identify arthropod species in the samples and calculate biodiversity-related indices, thereby assessing the impact of the transgenic crop on agricultural biodiversity.

As a further optimization of the present disclosure, the transgenic crop is transgenic maize or transgenic soybean.

As a further optimization of the present disclosure, the transgenic maize is DBN9229, and the transgenic soybean is DBN9004×DBN8002×DBN8205.

As a further optimization of the present disclosure, collecting rhizosphere soil samples of the transgenic crop includes: after emergence of the transgenic crop, performing diagonal five-point sampling, removing surface debris from the soil surface and collecting a soil sample from the rhizosphere soil layer at a depth of 2-15 cm at each sampling point, and mixing the soil samples from the five sampling points for DNA extraction; and
collecting phyllosphere surface attachment samples of the transgenic crop includes: during soil sampling, collecting one leaf each from upper, middle, and lower parts of a plant of the transgenic crop, thoroughly washing the leaves with ultrapure water, performing vacuum filtration using a vacuum filter and a microporous filter membrane with a pore size of 0.22 μm, and cutting the filter membrane into pieces for DNA extraction.

As a further optimization of the present disclosure, the FastDNA® Spin Kit for Soil is used to extract the DNA from the samples.

As a further optimization of the present disclosure, the amplification primers are an upstream primer mlCOIintF and a downstream primer jgHCO2198R for the mitochondrial cytochrome c oxidase subunit I (COI) gene, wherein the nucleotide sequences of the amplification primers are:
SEQ ID NO:1: upstream primer mlCOIintF: GGWACWGGWTGAACWGTWTAYCCYCC; and
SEQ ID NO:2: downstream primer jgHCO2198R: TAAACTTCAGGGTGACCAAARAAYCA.

The beneficial effects of the present disclosure are as follows:

(1) According to the method of the present disclosure, rhizosphere soil samples and phyllosphere surface attachment samples of a transgenic crop are collected during the same period. DNA is extracted from the samples. PCR amplification targeting the mitochondrial COI gene is then performed using the extracted DNA. The PCR amplification products are purified and subsequently subjected to HTS. The obtained OTU sequences are compared with the nt public database of the NCBI website to identify animal species and determine their relative abundance in the samples. Biodiversity-related indices are calculated based on these results to assess the impact of the transgenic crop on agricultural biodiversity. The present disclosure can effectively reduce the professional requirements and workload associated with field surveys of biodiversity. The method identifies a greater number of species more comprehensively and can achieve classification down to the species level. Furthermore, the method incurs relatively lower time and economic costs.

(2) According to the method of the present disclosure, high-accuracy detection of multi-trophic level organisms including soil collembolans and phyllosphere pollinating insects is achieved through simultaneous collection of rhizosphere soil samples and phyllosphere samples, combined with 313 base pair (bp) mitochondrial COI hypervariable region amplification and multi-database joint annotation. The present disclosure establishes a new paradigm for ecological risk assessment of transgenic crops, provides high-precision data support for environmental release decision-making of transgenic crops, sets a methodological benchmark for sustainable management of agricultural ecosystems, and promotes the advancement of biodiversity conservation from macroscopic observation to molecular-scale detection.

### BRIEF DESCRIPTION OF THE DRAWINGS

FIG. 1 is a table showing field survey results of arthropod species in tested soybean and maize materials by direct observation method.

FIG. 2 is a table showing field survey results of arthropod species in tested soybean and maize materials by HTS method.

FIG. 3 shows clustering of all biological species in maize and soybean fields (HTS method).

FIG. 4 shows hierarchical clustering analysis of maize and soybean samples at family level (HTS method).

FIG. 5 shows diversity indices of arthropods in different maize materials under field conditions (HTS method).

FIG. 6 shows diversity indices of arthropods in different soybean materials under field conditions (HTS method).

FIG. 7 shows Simpson index of arthropods in different maize materials under field conditions (HTS method).

FIG. 8 shows Simpson index of arthropods in different soybean materials under field conditions (HTS method).

FIG. 9 shows evenness index of arthropods in different maize materials under field conditions (HTS method).

FIG. 10 shows evenness index of arthropods in different soybean materials under field conditions (HTS method).

### DETAILED DESCRIPTION OF THE EMBODIMENTS

The following provides a detailed description of the present disclosure with reference to the accompanying drawings. It is to be expressly pointed out that the following specific embodiments are provided solely for further illustration of the present disclosure and are not to be construed as limiting the scope of protection thereof. Persons skilled in the art can make certain non-essential modifications and adjustments to the present disclosure based on the content described above.

### I. Materials

1. Transgenic maize DBN9229 and its non-transgenic comparator maize DBN567 were provided by Beijing Dabeinong Biotechnology Co., Ltd. The quality of all mentioned materials met the requirements specified for not less than Grade II maize seeds in GB 4404.1.

2. Transgenic soybean DBN9004×DBN8002×DBN8205 and its non-transgenic receptor JACK were provided by Beijing Dabeinong Biotechnology Co., Ltd.

3. Basta®, with glufosinate-ammonium as the active ingredient (content: 18%), formulated as a soluble concentrate (SL), was manufactured by Bayer CropScience, with Australia as the country of origin.

Unless otherwise specified, all methods used in the embodiment are conventional methods known to persons skilled in the art. All reagents and other materials, unless otherwise specified, were commercially available products.

### II. Methods

### 2.1 Experimental Design

Time of the experiment: April 2024-October 2024. Location of the experiment: Beijing Dabeinong Biotechnology Co., Ltd. base, Sijian Village, Chengxi Town, Lvyuan District, Changchun City, Jilin Province, China (43°56′12″N, 125° 15′07″E).

### 2.1.1 Maize

Experimental plots were arranged randomly. A 1.0 m wide isolation strip was set between plots. Each plot had an area of 150 m² (10 m×15 m). One plot served as one replicate, and each treatment was repeated 3 times. The experiment comprised 3 treatments:
Treatment 1: Transformation event DBN9229 sprayed with a glufosinate-ammonium containing herbicide (600 g active ingredient per hectare (g a.i./ha)), using the herbicide Basta®;
Treatment 2: Transformation event DBN9229 with manual weeding;
Treatment 3: Receptor variety, non-transgenic maize DBN567, with manual weeding.

Sowing time, sowing method, and seeding rate: Sowing was performed on April 27 using hill dropping. The conventional local seeding rate was used, and management followed conventional practices.

Herbicide application time: Application occurred on June 3, when the transformed maize had 3-4 trifoliate leaves.

### 2.1.2 Soybean

Experimental plots were arranged randomly. A 1.0 m wide isolation strip was set between plots. Each plot had an area of 150 m² (10 m×15 m). One plot served as one replicate, and each treatment was repeated 3 times. The experiment comprised 3 treatments:
Treatment 1: Transgenic soybean DBN9004×DBN8002×DBN8205 sprayed with a glufosinate-ammonium containing herbicide (600 g a.i./ha), using the herbicide Basta®;
Treatment 2: Transgenic soybean DBN9004×DBN8002×DBN8205 with manual weeding;
Treatment 3: Receptor variety, non-transgenic soybean Jack, with manual weeding.

Sowing time, sowing method, and seeding rate: Sowing was performed on May 8 using hill dropping. The conventional local seeding rate was used, and management followed conventional practices.

Herbicide application time: Application occurred on June 14, when the transgenic soybean had 3-4 trifoliate leaves.

### 2.2 Investigation on the Impact on Animal Diversity

### 2.2.1 Direct Observation Method

### (1) Maize Materials

The entire growth period of the transformed maize sprayed with glufosinate-ammonium, the transformed maize sprayed with water, and the receptor maize variety from the three treatment groups in step 2.1.1 was investigated using diagonal five-point sampling.

From emergence to maturity, investigations were conducted every 7 days. At each point, ten maize plants were fixed. Arthropod species and their developmental stages on the entire maize plant (recording aphids and spider mites on the upper, middle, and lower three leaves) and all arthropods on the ground were recorded. During the investigation, the number and species of main arthropods, including various insects and spiders, were recorded. Species that could not be identified in the field were numbered and brought back to the laboratory for identification. At the beginning of each investigation, the number of active and easily moving insects/spiders was quickly observed first.

Four indices of the arthropod community, including richness (number of species), diversity index, Simpson index, and evenness index, were used to analyze and compare the occurrence and development trends of the insect community, pest subcommunity, and natural enemy subcommunity in the field for each maize material. This was conducted to determine whether the transformation event and the application of glufosinate-ammonium to the transformed maize had an impact on the diversity of field arthropods.

### (2) Soybean Materials

The entire growth period of the transformed soybean sprayed with glufosinate-ammonium, the transformed soybean sprayed with water, and the receptor soybean variety from the three treatment groups in step 2.1.2 was investigated using diagonal five-point sampling.

From ten days after establishment to maturity, investigations were conducted every 10 days. At each point, ten soybean plants were fixed. All arthropod species and their quantities on the entire soybean plant were recorded. During the investigation, the number and species of main arthropods, including various insects and spiders, were recorded. Species that could not be identified in the field were numbered and brought back to the laboratory for identification. At the beginning of each investigation, the species and number of active and easily moving insects were quickly observed first.

Four indices of the arthropod community, including richness (number of species), diversity index, Simpson index, and evenness index, were used to analyze and compare the occurrence and development trends of the insect community, pest subcommunity, and natural enemy subcommunity in the field for each soybean material. This was conducted to determine whether the transgenic soybean and the application of glufosinate-ammonium to the transgenic soybean had an impact on the diversity of field arthropods.

### 2.2.2 High-Throughput Sequencing Method

### (1) Sample Collection and Processing

Sampling subjects: Soil and leaf samples were collected from each replicate plot of the six treatment groups mentioned in step 2.1.1 and step 2.2.2.

Sampling time: Sampling was performed once per month starting after crop emergence.

Sampling quantity: Diagonal five-point random sampling was adopted for each plot. Samples from the five points were mixed into one composite sample. For soil sampling, surface debris was removed from the soil surface. Approximately 200 g of rhizosphere soil at a depth of 2-15 cm was collected using a soil auger (approximately 1 kg per plot from five points) and placed into a sterile zip-lock bag. The bag was labeled with the material name, replicate number, and sampling time.

Simultaneously with soil sampling, at a location adjacent to each sampling point, one leaf each from the upper, middle, and lower parts of one soybean plant (15 leaves per plot from five points) and one leaf each from the upper, middle, and lower parts of one maize plant (15 leaves per plot from five points) were collected and placed into sterile zip-lock bags. Labeling information is shown in Table 1.

TABLE 1
Sample Labeling Information

| | | | Sampling | |
|---|---|---|---|---|
| Crop | Variety | Treatment | Type | Code |
| Maize | DBN9229 | Sprayed | Soil | MTG_S |
| | | with | Leaf | MTG_L |
| | | glyphosate | | |
| | DBN9229 | Manual | Soil | MTC_S |
| | | weeding | Leaf | MTC_L |
| | DBN567 | Manual | Soil | MCC_S |
| | | weeding | Leaf | MCC_L |
| | DBN9004 × | Sprayed | Soil | STG_S |
| | DBN8002 × | with | Leaf | STG_L |
| | DBN8205 | glyphosate | | |
| Soybean | DBN9004 × | Manual | Soil | STC_S |
| | DBN8002 × | weeding | Leaf | STC_L |
| | DBN8205 | Manual | Soil | SCC_S |
| | JACK | weeding | Leaf | SCC L |

Sample processing: For leaf samples, 200 mL of ultrapure water was added to the sterile zip-lock bag for each replicate. The leaves were thoroughly washed. Vacuum filtration was performed using a vacuum filter (JOANLAB) and a microporous filter membrane with a pore size of 0.22 μm. The filter membrane was then cut into pieces for DNA extraction. Soil samples were thoroughly mixed and then used for direct DNA extraction.

### (2) Sample DNA Extraction

DNA was extracted from the samples obtained in the aforementioned step (1) using the FastDNA® Spin Kit for Soil, in accordance with the kit's instructions. The purity and concentration of the DNA were detected using a NanoDrop2000 ultramicro spectrophotometer. The integrity of the DNA was detected by 1% agarose gel electrophoresis performed at 5 V/cm for 20 minutes.

### (3) PCR Amplification and High-Throughput Sequencing

Using the DNA extracted in the aforementioned step (2) as a template, PCR amplification was performed employing the upstream primer mlCOIintF and the downstream primer jgHCO2198R, which target the mitochondrial COI gene. The nucleotide sequences of the PCR amplification primers are:
SEQ ID NO:1: upstream primer mlCOIintF: GGWACWGGWTGAACWGTWTAYCCYCC; and
SEQ ID NO:2: downstream primer jgHCO2198R: TAAACTTCAGGGTGACCAAARAAYCA.

The PCR reaction system was as follows: 4 μL of 5× TransStart FastPfu Buffer, 2 μL of 2.5 mM deoxyribonucleotide triphosphates (dNTPs), 0.8 μL of upstream primer (5 μM), 0.8 μL of downstream primer (5 μM), 0.4 μL of TransStart FastPfu DNA Polymerase, 10 ng of template DNA, supplemented to a final volume of 20 μL.

The amplification program was as follows: pre-denaturation at 95° C. for 3 minutes; 27 cycles of denaturation at 95° C. for 30 seconds, annealing at 55° C. for 30 seconds, and extension at 72° C. for 30 seconds; followed by a stable extension at 72° C. for 10 minutes, and finally holding at 4° C. (PCR instrument: ABI GeneAmp® 9700).

The PCR products were recovered using 2% agarose gel electrophoresis. The recovered products were purified using a DNA Gel Recovery and Purification Kit (PCR Clean-Up Kit, YuHua, China). The purified recovery products were detected and quantified using a Qubit 4.0 Fluorometer (Thermo Fisher Scientific, USA).

The purified recovery products were sent to Shanghai Majorbio Bio-pharm Technology Co., Ltd. for HTS. Sequencing was performed using the Illumina PE300/PE250 platform. The obtained “reads” were assembled based on “overlap” relationships, and sequence quality was controlled and filtered. The nt public database of the NCBI website (ftp://ftp.ncbi.nlm.nih.gov/) was used for OTU sequence comparison, with a 97% sequence similarity threshold set as the standard for species annotation. Among the annotated OTU sequences, the kingdom Metazoa (encompassing all animals except protozoans) was selected as the OTU set for subsequent biodiversity analysis.

### 2.3 Statistical Analysis Results of the Investigation

### 2.3.1 Number of Species

All data analyses were performed on the Majorbio Cloud Platform (https://cloud.majorbio.com). Statistically annotated OTU species data were analyzed to determine the community composition of each sample at various taxonomic levels: class, order, family, and species. The Shannon diversity index, Simpson index, and Pielou's evenness index (Pielou_e) were calculated to analyze the biodiversity of each sample.

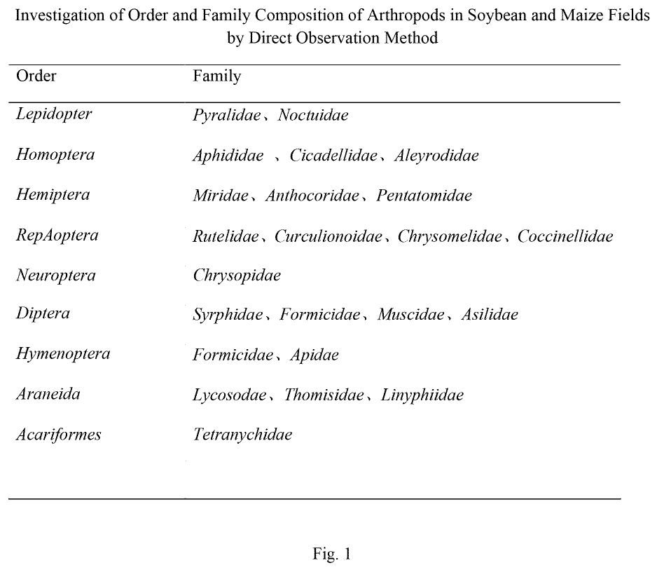

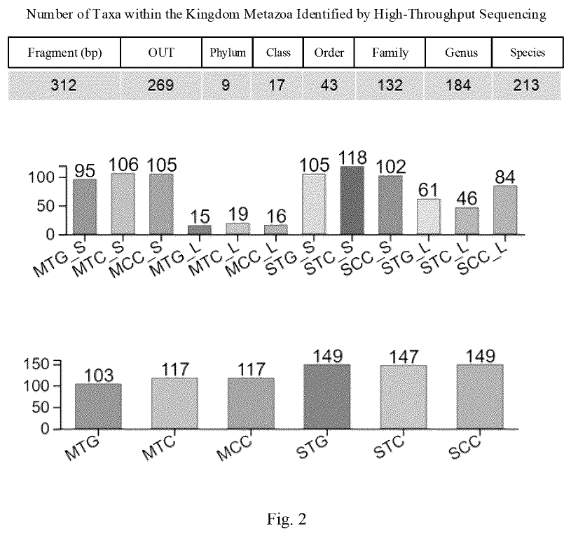

The present disclosure investigated the arthropod species in the fields of tested soybean and maize materials during the 2024 growth period using the direct observation method. The results revealed that the arthropod populations on soybean and maize plants comprised insects from 9 orders and 23 families (as shown in FIG. 1). Using the HTS method, a total of 213 species from 132 families were detected (as shown in FIG. 2).

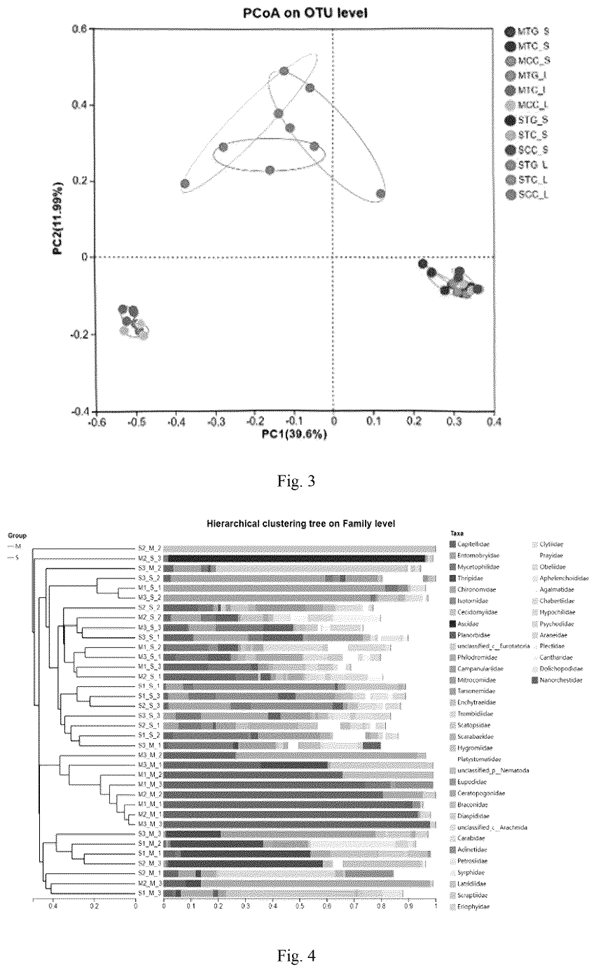

As shown in FIGS. 3 and 4, the soil samples yielded a high number of identified species with relatively small differences among them. In contrast, the leaf samples yielded a relatively lower number of identified species, but significant differences were observed between different crops. Furthermore, some species identified in the leaf samples were not detected in the soil samples. The sampling method had a considerable impact on species identification. The species identified in soil samples showed little variation across different crops, while those identified in leaf samples exhibited distinct differentiation among different crops. Therefore, when using the eDNA metabarcoding sequencing method to assess the impact of genetic modification on biodiversity, both soil and leaf sampling methods should be employed simultaneously.

### 2.3.2 Variation in Arthropod Community Diversity (Diversity Index)

The diversity index reflects the occurrence of arthropods in the farmland system and indicates the richness of arthropod species within the system. A higher diversity index denotes greater stability of the community. The diversity indices of arthropods in the fields of the receptor, the transformation event, and the transformation event sprayed with glufosinate-ammonium were calculated and analyzed from the survey data using the diversity index formula. The calculated diversity index values were retained to two decimal places. The diversity index formula is as follows:

$$H = - {\sum }_{i = 1}^{s} {P}_{i} Ln {P}_{i}$$

In the formula: H-Diversity index; Pi=Ni/N; Ni represents the number of individuals of the i-th species; N represents the total number of individuals.

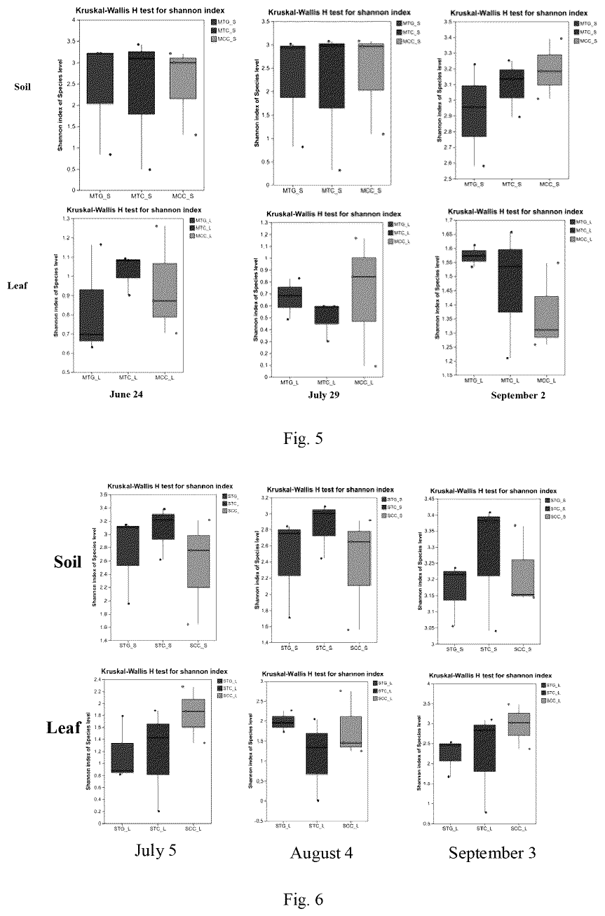

The calculated results from the survey data of field arthropod diversity indices for different maize materials, obtained by the direct observation method, are shown in Table 2; the calculated results from the survey data obtained by the HTS method are shown in FIG. 5.

TABLE 2
Diversity Indices of Field Arthropods in Different Maize Materials

| | Materials | | |
|---|---|---|---|
| | | | DBN9229 sprayed |
| | | | with glufosinate- |
| Survey Date | DBN567 | DBN9229 | ammonium |
| June 17 | 1.87 ± 0.10^{a} | 1.74 ± 0.06^{ab} | 1.66 ± 0.09^{b} |
| June 24 | 2.01 ± 0.01^{a} | 1.89 ± 0.06^{a} | 1.88 ± 0.09^{a} |
| July 1 | 2.09 ± 0.08^{a} | 2.04 ± 0.02^{a} | 2.03 ± 0.08^{a} |
| July 8 | 2.01 ± 0.05^{a} | 1.98 ± 0.10^{a} | 2.00 ± 0.13^{a} |
| July 15 | 2.20 ± 0.07^{a} | 2.20 ± 0.06^{a} | 2.15 ± 0.03^{a} |
| July 22 | 2.25 ± 0.10^{a} | 2.26 ± 0.12^{a} | 2.22 ± 0.16^{a} |
| July 29 | 2.46 ± 0.06^{a} | 2.47 ± 0.04^{a} | 2.46 ± 0.08^{a} |
| August 5 | 2.45 ± 0.03^{a} | 2.43 ± 0.07^{a} | 2.46 ± 0.10^{a} |
| August 12 | 2.26 ± 0.12^{a} | 2.25 ± 0.07^{a} | 2.25 ± 0.07^{a} |
| August 19 | 2.19 ± 0.13^{a} | 2.18 ± 0.19^{a} | 2.21 ± 0.17^{a} |
| August 26 | 2.20 ± 0.06^{a} | 2.17 ± 0.05^{a} | 2.19 ± 0.15^{a} |
| September 2 | 2.10 ± 0.06^{a} | 2.13 ± 0.04^{a} | 2.11 ± 0.09^{a} |
| September 9 | 1.88 ± 0.09^{a} | 1.93 ± 0.14^{a} | 1.91 ± 0.07^{a} |
| September 16 | 1.76 ± 0.06^{a} | 1.81 ± 0.10^{a} | 1.77 ± 0.12^{a} |

Note:
Values within a row followed by the different lowercase letter are significantly different at p < 0.05 according to Tukey's multiple range test. The same below.

The results in Table 2 above indicate that the diversity index of arthropods in the farmland system was higher in August. During some survey periods, the diversity index of arthropods in the receptor was significantly higher than that in the transformation event treated with herbicide, primarily due to the lower baseline number of insects during the initial or final survey periods and the presence of target Lepidoptera pests infesting the receptor. For the remaining survey periods, no significant differences were observed in the arthropod diversity indices between the receptor and the transformation event treatments. This indicates that, compared to the receptor DBN567, the transformation event and the transformation event sprayed with glufosinate-ammonium had no adverse effects on the variation in field arthropod diversity.

As shown in FIG. 5, the HTS results revealed no significant differences in the arthropod diversity indices between the receptor and the transformation event treatments on June 24, July 29, and September 2. These results are consistent with those obtained by the direct observation method, demonstrating that the HTS method can be used for detecting and calculating animal diversity in crop fields.

The field arthropod diversity indices for the receptor, the transgenic soybean, and the transgenic soybean sprayed with glufosinate-ammonium were calculated and analyzed through the aforementioned diversity index formula applied to the survey data. The calculated results from the direct observation method are shown in Table 3, and the calculated results from the survey data obtained by the HTS method are shown in FIG. 6.

TABLE 3
Diversity Indices of Field Arthropods
in Different Soybean Materials

| | Materials | | |
|---|---|---|---|
| | | | Transgenic Soybean |
| | | Transgenic | sprayed with |
| Survey Date | Jack | Soybean | glufosinate-ammonium |
| June 25 | 1.87 ± 0.07^{a} | 1.77 ± 0.11^{a} | 1.74 ± 0.06^{a} |
| July 5 | 2.17 ± 0.06^{a} | 2.00 ± 0.05^{b} | 1.98 ± 0.01^{b} |
| July 15 | 2.44 ± 0.03^{a} | 2.40 ± 0.04^{a} | 2.38 ± 0.05^{a} |
| July 25 | 2.56 ± 0.09^{a} | 2.51 ± 0.08^{a} | 2.51 ± 0.03^{a} |
| August 4 | 2.40 ± 0.09^{a} | 2.34 ± 0.06^{a} | 2.32 ± 0.16^{a} |
| August 14 | 2.34 ± 0.07^{a} | 2.31 ± 0.10^{a} | 2.32 ± 0.02^{a} |
| August 24 | 2.28 ± 0.07^{a} | 2.23 ± 0.10^{a} | 2.26 ± 0.08^{a} |
| September 3 | 2.35 ± 0.02^{a} | 2.34 ± 0.07^{a} | 2.34 ± 0.09^{a} |
| September 13 | 2.35 ± 0.06^{a} | 2.33 ± 0.11^{a} | 2.39 ± 0.05^{a} |
| September 23 | 2.17 ± 0.13^{a} | 2.23 ± 0.09^{a} | 2.18 ± 0.18^{a} |

The results in Table 3 above indicate that the diversity index of arthropods in the farmland system was higher in late July. During some survey periods, the diversity index of arthropods in the receptor was significantly higher than that in the two transgenic soybean treatments, primarily due to the lower baseline number of insects during the initial survey periods and the presence of target Lepidoptera pests infesting the receptor. For the remaining survey periods, no significant differences were observed in the arthropod diversity indices between the receptor and the two transgenic soybean treatments. This indicates that, compared to the receptor Jack, the transgenic soybean and the transgenic soybean sprayed with glufosinate-ammonium had no adverse effects on the variation in field arthropod diversity.

As shown in FIG. 6, the HTS results revealed no significant differences in the arthropod diversity indices between the receptor and the transformation event treatment groups on July 5, August 4, and September 3. These results are consistent with those obtained by the direct observation method, demonstrating that the HTS method can be used for detecting and calculating animal diversity in soybean crop fields.

### 2.3.3 Variation in Arthropod Community Dominance Concentration (Simpson Index)

The Simpson index (dominance index) reflects the dominance of arthropods within the entire community. The field arthropod Simpson indices for the receptor, the transformation event, and the transformation event sprayed with glufosinate-ammonium were calculated and analyzed through the Simpson index formula applied to the survey data. The calculated Simpson index values were retained to two decimal places. The Simpson index formula is as follows:

$$C = {\sum }_{1}^{n} {( {N}_{i} / N )}^{2} .$$

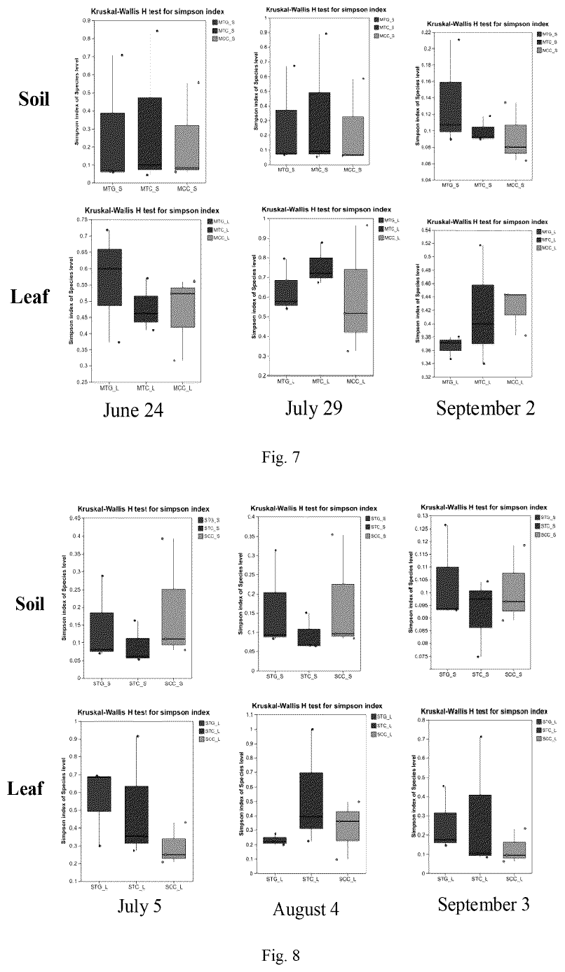

In the formula: C—Simpson index; Ni represents the number of individuals of the i-th species; N represents the total number of individuals. The calculated results from the survey data of field arthropod Simpson indices for different maize materials, obtained by the direct observation method, are shown in Table 4; the calculated results from the survey data obtained by the HTS method are shown in FIG. 7.

TABLE 4
Simpson Indices of Field Arthropods in Different Maize Materials

| | Materials | | |
|---|---|---|---|
| | | | DBN9229 sprayed |
| | | | with glufosinate- |
| Survey Date | DBN567 | DBN9229 | ammonium |
| June 17 | 0.18 ± 0.02^{a} | 0.19 ± 0.01^{a} | 0.21 ± 0.02^{a} |
| June 24 | 0.16 ± 0.01^{a} | 0.17 ± 0.01^{a} | 0.18 ± 0.01^{a} |
| July 1 | 0.14 ± 0.01^{a} | 0.15 ± 0.00^{a} | 0.15 ± 0.01^{a} |
| July 8 | 0.22 ± 0.02^{a} | 0.22 ± 0.04^{a} | 0.21 ± 0.05^{a} |
| July 15 | 0.18 ± 0.03^{a} | 0.18 ± 0.02^{a} | 0.19 ± 0.01^{a} |
| July 22 | 0.18 ± 0.03^{a} | 0.18 ± 0.03^{a} | 0.20 ± 0.05^{a} |
| July 29 | 0.13 ± 0.01^{a} | 0.13 ± 0.01^{a} | 0.14 ± 0.02^{a} |
| August 5 | 0.13 ± 0.01^{a} | 0.14 ± 0.02^{a} | 0.13 ± 0.02^{a} |
| August 12 | 0.20 ± 0.03^{a} | 0.20 ± 0.02^{a} | 0.20 ± 0.02^{a} |
| August 19 | 0.21 ± 0.04^{a} | 0.21 ± 0.06^{a} | 0.20 ± 0.04^{a} |
| August 26 | 0.20 ± 0.02^{a} | 0.20 ± 0.02^{a} | 0.20 ± 0.04^{a} |
| September 2 | 0.20 ± 0.02^{a} | 0.19 ± 0.01^{a} | 0.19 ± 0.02^{a} |
| September 9 | 0.24 ± 0.03^{a} | 0.23 ± 0.03^{a} | 0.23 ± 0.02^{a} |
| September 16 | 0.28 ± 0.02^{a} | 0.26 ± 0.04^{a} | 0.27 ± 0.04^{a} |

The results in Table 4 above indicate that no significant differences were observed in the arthropod Simpson indices between the receptor and the transformation event treatments at the same survey time. Compared to the receptor DBN567, the transformation event and the transformation event sprayed with glufosinate-ammonium had no adverse effects on the variation in field arthropod dominance concentration.

As shown in FIG. 7, the HTS results revealed no significant differences in the arthropod Simpson indices between the receptor and the transformation event treatments on June 24, July 29, and September 2. These results are consistent with those obtained by the direct observation method, demonstrating that the HTS method can be used for detecting and calculating animal diversity in crop fields.

The field arthropod Simpson indices for the receptor, the transgenic soybean, and the transgenic soybean sprayed with glufosinate-ammonium were calculated and analyzed through the aforementioned Simpson index formula applied to the survey data. The calculated results from the direct observation method are shown in Table 5, and the calculated results from the survey data obtained by the HTS method are shown in FIG. 8.

TABLE 5
Simpson Indices of Field Arthropods
in Different Soybean Materials

| | Materials | | |
|---|---|---|---|
| | | | Transgenic Soybean |
| | | Transgenic | sprayed with |
| Survey Date | Jack | Soybean | glufosinate-ammonium |
| June 25 | 0.18 ± 0.01^{a} | 0.19 ± 0.03^{a} | 0.19 ± 0.02^{a} |
| July 5 | 0.13 ± 0.01^{a} | 0.15 ± 0.00^{a} | 0.15 ± 0.00^{a} |
| July 15 | 0.10 ± 0.01^{a} | 0.10 ± 0.01^{a} | 0.10 ± 0.01^{a} |
| July 25 | 0.12 ± 0.02^{a} | 0.12 ± 0.01^{a} | 0.12 ± 0.01^{a} |
| August 4 | 0.17 ± 0.02^{a} | 0.17 ± 0.02^{a} | 0.18 ± 0.05^{a} |
| August 14 | 0.18 ± 0.02^{a} | 0.18 ± 0.03^{a} | 0.18 ± 0.00^{a} |
| August 24 | 0.20 ± 0.03^{a} | 0.20 ± 0.03^{a} | 0.19 ± 0.02^{a} |
| September 3 | 0.16 ± 0.01^{a} | 0.16 ± 0.02^{a} | 0.16 ± 0.03^{a} |
| September 13 | 0.13 ± 0.02^{a} | 0.14 ± 0.03^{a} | 0.12 ± 0.01^{a} |
| September 23 | 0.16 ± 0.04^{a} | 0.15 ± 0.03^{a} | 0.16 ± 0.05^{a} |

The results in Table 5 above indicate that no significant differences were observed in the arthropod Simpson indices between the receptor and the transgenic soybean treatments at the same survey time. Compared to the receptor Jack, the transgenic soybean and the transgenic soybean sprayed with glufosinate-ammonium had no adverse effects on the variation in field arthropod dominance concentration.

As shown in FIG. 8, the HTS results revealed no significant differences in the arthropod Simpson indices between the receptor and the transformation event treatments on July 5, August 4, and September 3. These results are consistent with those obtained by the direct observation method, demonstrating that the HTS method can be used for detecting and calculating animal diversity in crop fields.

### 2.3.4 Variation in Arthropod Community Evenness (Evenness Index)

The evenness index reflects the uniformity of species quantities within an arthropod population. The field arthropod evenness indices for the receptor, the transformation event, and the transformation event sprayed with glufosinate-ammonium were calculated and analyzed through the evenness index formula applied to the survey data. The calculated evenness index values were retained to two decimal places. The evenness index formula is as follows:

$$J = H / ln S .$$

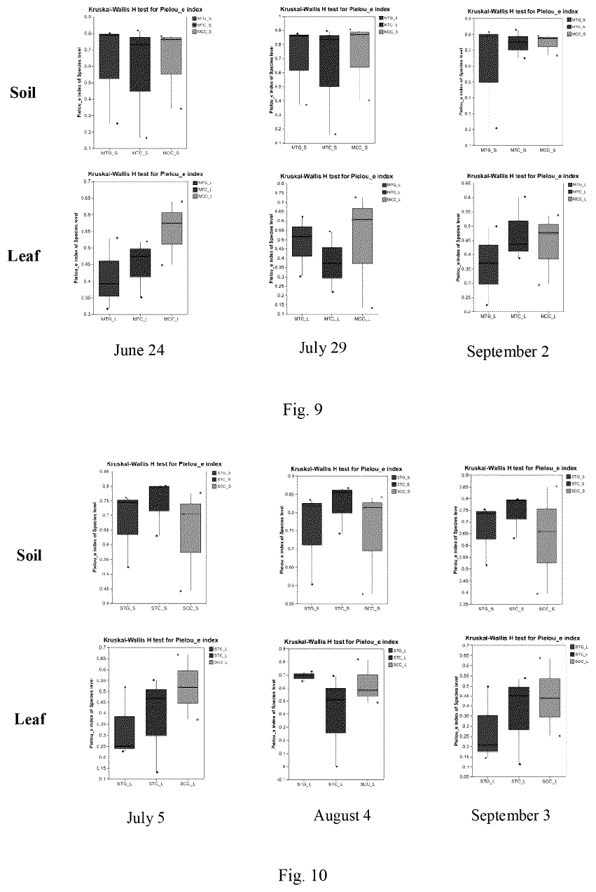

In the formula: J—Evenness index; H—Diversity index; S—Number of species. The calculated results from the survey data of field arthropod evenness indices for different maize materials, obtained by the direct observation method, are shown in Table 6; the calculated results from the survey data obtained by the HTS method are shown in FIG. 9.

TABLE 6
Evenness Indices of Field Arthropods in Different Maize Materials

| | Materials | | |
|---|---|---|---|
| | | | DBN9229 sprayed |
| | | | with glufosinate- |
| Survey Date | DBN567 | DBN9229 | ammonium |
| June 17 | 0.87 ± 0.02^{a} | 0.92 ± 0.01^{a} | 0.90 ± 0.01^{a} |
| June 24 | 0.89 ± 0.03^{a} | 0.93 ± 0.02^{a} | 0.89 ± 0.03^{a} |
| July 1 | 0.89 ± 0.02^{a} | 0.90 ± 0.02^{a} | 0.90 ± 0.03^{a} |
| July 8 | 0.75 ± 0.02^{a} | 0.76 ± 0.04^{a} | 0.76 ± 0.04^{a} |
| July 15 | 0.79 ± 0.03^{a} | 0.81 ± 0.02^{a} | 0.80 ± 0.01^{a} |
| July 22 | 0.79 ± 0.02^{a} | 0.80 ± 0.02^{a} | 0.79 ± 0.05^{a} |
| July 29 | 0.81 ± 0.02^{a} | 0.81 ± 0.01^{a} | 0.82 ± 0.04^{a} |
| August 5 | 0.81 ± 0.01^{a} | 0.80 ± 0.02^{a} | 0.80 ± 0.03^{a} |
| August 12 | 0.70 ± 0.04^{a} | 0.71 ± 0.02^{a} | 0.71 ± 0.02^{a} |
| August 19 | 0.67 ± 0.04^{a} | 0.68 ± 0.06^{a} | 0.70 ± 0.05^{a} |
| August 26 | 0.71 ± 0.03^{a} | 0.70 ± 0.02^{a} | 0.71 ± 0.06^{a} |
| September 2 | 0.73 ± 0.02^{a} | 0.74 ± 0.01^{a} | 0.76 ± 0.03^{a} |
| September 9 | 0.73 ± 0.04^{a} | 0.75 ± 0.05^{a} | 0.75 ± 0.03^{a} |
| September 16 | 0.74 ± 0.01^{a} | 0.76 ± 0.04^{a} | 0.75 ± 0.04^{a} |

The results in Table 6 above indicate that no significant differences were observed in the arthropod evenness indices between the receptor and the transformation event treatments at the same survey time. Compared to the receptor DBN567, the transformation event and the transformation event sprayed with glufosinate-ammonium had no adverse effects on the variation in field arthropod evenness.

As shown in FIG. 9, the HTS results revealed no significant differences in the arthropod evenness indices between the receptor and the transformation event treatments on June 24, July 29, and September 2. These results are consistent with those obtained by the direct observation method, demonstrating that the HTS method can be used for detecting and calculating animal diversity in crop fields.

The field arthropod evenness indices for the receptor, the transgenic soybean, and the transgenic soybean sprayed with glufosinate-ammonium were calculated and analyzed through the aforementioned evenness index formula applied to the survey data. The calculated results from the direct observation method are shown in Table 7, and the calculated results from the survey data obtained by the HTS method are shown in FIG. 10.

TABLE 7
Evenness Indices of Field Arthropods
in Different Soybean Materials

| | Materials | | |
|---|---|---|---|
| | | | Transgenic Soybean |
| | | Transgenic | sprayed with |
| Survey Date | Jack | Soybean | glufosinate-ammonium |
| June 25 | 0.90 ± 0.02^{a} | 0.93 ± 0.02^{a} | 0.95 ± 0.04^{a} |
| July 5 | 0.92 ± 0.01^{a} | 0.94 ± 0.01^{a} | 0.92 ± 0.03^{a} |
| July 15 | 0.93 ± 0.01^{a} | 0.94 ± 0.02^{a} | 0.93 ± 0.02^{a} |
| July 25 | 0.84 ± 0.03^{a} | 0.85 ± 0.02^{a} | 0.85 ± 0.02^{a} |
| August 4 | 0.79 ± 0.02^{a} | 0.78 ± 0.02^{a} | 0.78 ± 0.06^{a} |
| August 14 | 0.77 ± 0.02^{a} | 0.77 ± 0.04^{a} | 0.77 ± 0.01^{a} |
| August 24 | 0.73 ± 0.03^{a} | 0.76 ± 0.03^{a} | 0.76 ± 0.02^{a} |
| September 3 | 0.83 ± 0.01^{a} | 0.84 ± 0.03^{a} | 0.84 ± 0.03^{a} |
| September 13 | 0.88 ± 0.03^{a} | 0.88 ± 0.05^{a} | 0.89 ± 0.01^{a} |
| September 23 | 0.86 ± 0.04^{a} | 0.86 ± 0.02^{a} | 0.84 ± 0.06^{a} |

The results in Table 7 above indicate that no significant differences were observed in the arthropod evenness indices between the receptor and the transgenic soybean treatments at the same survey time. This indicates that, compared to the receptor Jack, the transgenic soybean and the transgenic soybean sprayed with glufosinate-ammonium had no adverse effects on the variation in field arthropod evenness.

As shown in FIG. 10, the HTS results revealed no significant differences in the arthropod evenness indices between the receptor and the transformation event treatments on July 5, August 4, and September 3. These results are consistent with those obtained by the direct observation method, demonstrating that the HTS method can be used for detecting and calculating animal diversity in crop fields.

The above embodiments merely represent several implementation modes of the present disclosure, and the description thereof is relatively specific and detailed, but shall not be construed as limiting the scope of the patent of the present disclosure. It should be pointed out that a person of ordinary skill in the art may make several modifications and improvements without departing from the concept of the present disclosure, and these all fall within the protection scope of the present disclosure.

What is claimed is:

1. A method for assessing an impact of a transgenic plant on agricultural biodiversity using environmental DNA (eDNA) metabarcoding sequencing, comprising the following steps:
collecting rhizosphere soil samples and phyllosphere surface attachment samples of a transgenic crop during the same period, and extracting deoxyribonucleic acid (DNA) from the samples;
using the extracted DNA as a template, performing polymerase chain reaction (PCR) amplification with amplification primers; and
purifying PCR amplification products, subjecting the purified PCR amplification products to high-throughput sequencing (HTS) to obtain sequences, comparing the obtained operational taxonomic unit (OTU) sequences with the nucleotide (nt) public database on the website of the National Center for Biotechnology Information (NCBI) to identify arthropod species in the samples and calculate biodiversity-related indices, thereby assessing the impact of the transgenic crop on agricultural biodiversity.

2. The method for assessing the impact of the transgenic plant on agricultural biodiversity using eDNA metabarcoding sequencing according to claim 1, wherein the transgenic crop is transgenic maize or transgenic soybean.

3. The method for assessing the impact of the transgenic plant on agricultural biodiversity using eDNA metabarcoding sequencing according to claim 2, wherein the transgenic maize is DBN9229, and the transgenic soybean is DBN9004×DBN8002× DBN8205.

4. The method for assessing the impact of the transgenic plant on agricultural biodiversity using eDNA metabarcoding sequencing according to claim 1, wherein collecting rhizosphere soil samples of the transgenic crop comprises: after emergence of the transgenic crop, performing diagonal five-point sampling, removing surface debris from the soil surface and collecting a soil sample from the rhizosphere soil layer at a depth of 2-15 cm at each sampling point, and mixing the soil samples from the five sampling points for DNA extraction; and
collecting phyllosphere surface attachment samples of the transgenic crop comprises: during soil sampling, collecting one leaf each from upper, middle, and lower parts of a plant of the transgenic crop, thoroughly washing the leaves with ultrapure water, performing vacuum filtration using a vacuum filter and a microporous filter membrane with a pore size of 0.22 μm, and cutting the filter membrane into pieces for DNA extraction.

5. The method for assessing the impact of the transgenic plant on agricultural biodiversity using eDNA metabarcoding sequencing according to claim 1, wherein the FastDNA® Spin Kit for Soil is used to extract the DNA from the samples.

6. The method for assessing the impact of the transgenic plant on agricultural biodiversity using eDNA metabarcoding sequencing according to claim 1, wherein the amplification primers are an upstream primer mlCOIintF and a downstream primer jgHCO2198R for the mitochondrial cytochrome c oxidase subunit I (COI) gene, wherein the nucleotide sequences of the amplification primers are:
SEQ ID NO:1: upstream primer mlCOIintF: GGWACWGGWTGAACWGTWTAYCCYCC; and
SEQ ID NO:2: downstream primer jgHCO2198R: TAAACTTCAGGGTGACCAAARAAYCA.


SEQUENCE LISTING (2 sequences)

SEQ ID NO: 1 - DNA, 26 bp, synthetic construct
  source 1..26: mol_type = other DNA
ggwacwggwtgaacwgtwtayccycc

SEQ ID NO: 2 - DNA, 26 bp, synthetic construct
  source 1..26: mol_type = other DNA
taaacttcagggtgaccaaaraayca